(19) United States
(12) Patent Application Publication
(10) Pub. No.: US 2021/0045645 A1
(43) Pub. Date: Feb. 18, 2021

(54) METHOD AND SYSTEM FOR MAPPING BRAIN DYSFUNCTION FOR PSYCHIATRIC AND NEUROLOGICAL DISORDERS

(71) Applicant: Dorian Aur, London (CA)

(72) Inventor: Dorian Aur, London (CA)

(21) Appl. No.: 16/538,999

(22) Filed: Aug. 13, 2019

(51) Int. Cl. A61B 5/04; A61B 5/0476; A61B 5/00; G16H 20/10; G16H 50/50

(52) U.S. Cl. CPC A61B 5/04012; A61B 5/0476; A61B 5/72; A61B 5/4082; G16H 50/50; A61B 5/4094; A61B 5/4088; G16H 20/10

(57) ABSTRACT

A brain mapping system and methods for providing personalized therapy for a broad range of brain dysfunctions by determining the location and the extent of the brain regions that have to be therapeutically targeted in each subject. The present invention includes means to record specific characteristics of brain activity, detect and display brain regions that present signatures of disease or dysfunctions by using a computing system. The therapy is tuned to target detected brain regions to restore specific connectivity characteristics using invasive, non-invasive stimulation, neurofeedback or drug administration. While connectivity characteristics are estimated based on resting state recordings the therapy will be performed in successive steps to alter network fragmentation in dysfunctional brain regions. The improved treatment is tailored to individual patients that will learn how to reshape specific connectivity characteristics to target the determined location and the extent of brain regions and maximize the therapeutic potential. The brain mapping technology is suited for different technologies and not limited to electroencephalography (EEG) or magneto electroencephalography (MEG).

### CROSS-REFERENCE TO RELATED APPLICATIONS

This application claims priority from U.S. Provisional Patent Application 62/723,761 filed Aug. 28, 2018, which is incorporated herein by reference.

### FIELD OF THE INVENTION

The present invention relates to the field of brain mapping. In particular, the invention relates to mapping brain dysfunctions before and after therapy for different brain conditions and treatments.

### BACKGROUND OF THE INVENTION

Whole head quantitative electroencephalography (qEEG) can be used for multiple purposes to detect changes in brain health, monitor the effects of therapy or provide neurofeedback training systems. Clinical application of topographic electroencephalography (EEG) mapping methods is still limited after almost 60 years of research even the progress in this field is remarkable (Klotz et al., 1993; Fischell et la., 2001; Acharya et al., 2018).

Different software packages (eConnectome, MNE and sLORETA, Neuroguide, WinEEG) are already used to map changes that occur in case of traumatic brain injury (TBI), posttraumatic stress disorder (PTSD) and major depressive disorders (MDD), or to localize the epileptogenic networks that lead to seizure occurrence. These conditions can be linked to suicidal ideation and the development of dementia later in life (Mohlenhoff et al., 2017) while mild grades of concussion may lead to disability or even death.

Having accurate brain maps that show the location of brain dysfunctions can help neurologists and psychiatrists to figure out the optimal therapy for different brain conditions. Electroencephalographic brain recordings were proposed to identify the regions that needed to be targeted by therapy for various neurological disorders, see U.S. Pat. Nos. 5,024,235, 5,406,957, and 8,239,014. The localization of electrical activity is performed by spatial filtering that generates a neural activity index map (Van Veen et al., 1997), source reconstruction using dipoles, distributed sources and beamformers (Michel et al., 2004, Oostenveld et al., 2011). Various other techniques were used to compute minimum norm estimates (LORETA, sLORETA, S-MAP) for non-parametric methods and beamforming techniques, BESA, and subspace techniques such as MUSIC (Grech et al., 2008).

All the above techniques have serious problems, however these issues have been only recently recognized (Anzolin et al., 2019). The inverse EEG solution has low resolution due to the volume conduction and noise with direct impact on the accuracy of the source analysis and led to mixing effects (Biscay et al., 2018). The alteration of dominant brain frequencies has limited therapeutic potential since the change of dominant brain frequencies is not necessarily related to brain dysfunctions or mental disorders (see, U.S. Pat. Nos. 8,082,031, 9,486,639, 9,460,400).

Continuous interactions between all brain regions are required for normal brain function (Aur and Jog, 2010; Aur et al., 2011; Aur and Tuszynski, 2017). As administered today the therapy for MDD and related brain disorders is ineffective in over 60% of patients (Paris, 2014).

In addition, each brain is substantially different and the response to therapy can be diverse. The location and the extent of the brain regions that have to be stimulated vary and are dependent on the patient's condition. None of known patents (e.g. Stubbeman and William 2013: U.S. Pat. Nos. 5,024,235; 5,406,957; 8,239,014) except U.S. Pat. No. 8,600,513 are tailored to determine specific characteristics for individual patients. The location and the extent of the brain regions that have to be stimulated are the most important characteristics required to shape neurofeedback systems, non-invasive or invasive techniques such deep brain stimulation (DBS). However, in all proposed solutions such characteristics are not determined in the individual subjected to therapy. Many techniques pick the noise increase and report as brain activity. All the above issues are known (well, son of) and are not solved by previous patents.

In addition, the measurement of brain drug concentrations and their effects is currently in infancy. Therapeutic drug monitoring (TDM) of serum concentrations is the only available means of estimating drug levels in the brain (Mitchell, 2000, Wang et al., 2015). The technique is less precise since drug levels are detected in the plasma not in the brain. How much the drug penetrates the blood-brain barrier in each patient is currently unknown. Nuclear magnetic resonance spectroscopy (NMS) estimates drug concentrations only in small brain regions, it is highly expensive and imprecise due to noise levels.

Despite several solutions proposed in the past, the brain mapping techniques either are unable to fully detect brain regions with network dysfunctions or have poor resolution. The system and the method proposed in this patent overcomes the above issues.

### SUMMARY OF THE INVENTION

The present disclosure describes an invention that has a number of embodiments that can be applied in combination with one another but may also have individual application.

The mapping procedure requires the subject to be connected to an EEG system that records electrical activity of the brain either from the scalp or from intracranially implanted electrodes.

In one embodiment, there is provided a system and methods for brain mapping of a subject, the system comprising: a means for recording brain activity; a computing means, wherein the computing means is capable of identifying dysfunctional network patterns of traumatic brain injury in a subject based on recorded electroencephalographic EEG activity. The network pattern includes a topographical map of network fragmentation where computed nonlinear complexity measures are mapped and displayed for specific brain regions. Network dysfunctions (injuries) are located in the regions with abnormally increased network fragmentation (Aur et al., 2018; Aur and Jog, 2019). Network fragmentation maps (NFMs) are representations of normalized network fragmentation values that directly provide the brain regions with network brain dysfunctions or network injury. No reference to a control group is needed to compute or analyze normalized network fragmentation in single subjects.

In one embodiment NFMs are used to compare control subjects and patient with traumatic brain injuries.

In another embodiment NFMs are used to identify and detect the location of dysfunctional or injured networks in MDD. Personalized therapy may include noninvasive, invasive stimulation and/or drug therapy.

In another embodiment. NFMs are used to monitor and evaluate the effect of drug therapy in each brain region and the occurrence of side effects.

In another embodiment network fragmentation maps are used detect the location of dysfunctional, injured network regions within subcortical structure. Brain mapping report makes it easier to visualize and identify dysfunctional brain regions with network injuries before and after therapy in major depression, epilepsy, traumatic brain injury (TBI). multiple sclerosis, tinnitus, autism spectrum disorders (ASD), Parkinson, dyskinesia, Alzheimer's disease, neuropathic pain or chronic migraine. NEMs indicate which brain regions have to be targeted by therapy and the therapy effects can be monitored so either drugs or non-invasive stimulation can be used.

The foregoing general description and detailed description below are exemplary, but are not restrictive, of the invention.

### BRIEF DESCRIPTION OF THE DRAWINGS

The accompanying drawings illustrate non-limiting examples of the present invention.

FIG. 1 is a general schematic illustration showing a system of the present invention according to an example implementation. The system includes: an electroencephalograph (EE) 100. a computing system 110 equipped with wireless transmission of information connected to a portable personal computer 120.

FIG. 2 shows the drop of symptom severity between 1-week post TBI 210 and 1-year post TBI, 220. The decrease of maximum value of network fragmentation is statistically significantly correlated with the reduction of symptom severity 230. Sorted values of percentage increase of maximum value of network fragmentation one year after TBI are shown for each subject 240. The reduction of symptom severity of each subject one year post-TBI is represented by bars 250.

FIG. 3 displays topographic map of mean network fragmentation of control subjects 310 and subject AB with traumatic brain injury at 1-week 320 and one year post-TBI 330. At 1-week after TBI, the presence of network injuries can be observed in the frontopolar 340 and supraorbital regions including left DLPFC 350. One year post-TBt the increase of network fragmentation with 29% in the left DLPFC 360 and orbitofrontal regions 370 inhibits functional recovery. Symptom severity increases from 3 to 4 one year post concussion.

FIG. 4 displays a technique to locate the extension of network injury 410 and determine the positioning of the built-on electrodes 420 to provide non-invasive stimulation. In order to maximize treatment benefit of non-invasive stimulation one electrode needs to be in the focal point of the network injury 430. The generated current by the non-invasive system will flow inside the brain between the electrode located in the middle 430 and other electrodes 420 and target the regions with abnormal network fragmentation. In general the electrodes can have different locations which may include the right or left shoulder, right supraobital ridge or spinal cord (see, Lenoir et al., 2018)

In one embodiment healthy people (control subjects) are used to provide a basis for comparison.

FIG. 5 displays a topographic view of normalized network fragmentation of control subjects 510. This fingerprint of network fragmentation in healthy control subjects defines the Innate resting-state Network Fragmentation (INF). The brain with network dysfunctions (injuries) shows a different network pattern, see 520. At baseline, before therapy NFM of subject with mild depression (HDRS=17) presented in FIG. 5 shows increased network fragmentation 520 in the left dorsolateral prefrontal region (DLPFC). The network dysfunction (injury) located in the left DLPFC 530 is displayed in dark gray color. HDRS-17 is the 17-item Hamilton Depression Rating Scale.

FIG. 6 displays NFM of the selected remitter with mild depression (HDRS=17) before therapy at baseline. The presence of network dysfunction (injury) can be observed in dark grey color in the left dorsolateral prefrontal cortex 610. Three months later after non-invasive stimulation, the network injury cannot be detected in the left DLPFC see the light grey color at F3 site 620. The subject is in remission, depression symptoms were lifted after non-invasive stimulation (HDRS=2).

FIG. 7 displays a baseline topographic map of normalized network fragmentation of a selected non-remitter. Network injuries are shown in dark grey color at baseline in the left parieto-temporal region 710 and right parieto-temporal region 720. The severity of depression decreases from MADRS=35 at baseline to MADRS=27 after two weeks of drug therapy. A drop of network fragmentation and a slight shift of network dysfunction (injury) to parietal regions 730 and 740 can be observed. After 8 weeks of escitalopram therapy the severity of depression drops further (MADRS=14) and network injury has shifted to the right primary motor cortex 750. Drug therapy can shift the location of brain dysfunctions (injuries) to other regions which lead to different symptoms.

FIG. 8 displays a topographic plot of F-test statistic of linear regression that shows specific brain regions in dark grey color in the left DLPFC 810, right primary motor region 820 where there is a statistically significant relationship between changes of network fragmentation and the drop of symptom severity eight weeks after escitalopram administration. A decrease of depression symptoms occurs in the next two months after escitalopram administration if network dysfunctions are located in the brain regions with high positive regression coefficients represented in dark gray color in the left DLPFC 830 and an increase of depression symptoms can be observed in patients with network dysfunctions located in brain regions with high negative values of regression coefficients represented in light grey color in the right primary motor cortex 840.

FIG. 9 displays a topographic representation of network fragmentation of a selected non-remitter with severe depression (HDRS=25) at baseline. The injured networks can be observed in the right DLPFC 910, right occipito-parietal region 920, see the dark grey region at electrodes: F4, Fc4, P4 and O2 and left primary motor region 930. If FDA-approved therapeutic is performed, transcranial magnetic therapy (TMS) targets the left DLPFC which is a healthy region. Three months after stimulation dysfunctional (injured) networks can be detected in the same brain regions in the right DLPFC 940, right occipito-parietal region 950, see the dark grey region at electrodes: F4, Fc4, P4 and O2 and left primary motor region 960. The stimulation of the healthy region (left DLPFC at F3 site) has worsened the severity of depression (HDRS=31).

FIG. 10 displays topographic maps of network fragmentation of a selected non-remitter with severe depression (HDRS=25) at baseline 1010, week 2, 1030 and week 8, 1050 of antidepressant therapy. The brain map presented corresponds to the maximal negative coefficient values (detected by statistical analyses shown in FIG. 8. The network (dysfunction) injury is located in the primary motor cortex, right hemisphere, at electrode C4. After escitalopram administration the severity of depression increases from MADRS=22 at baseline to MADRS=24. The topographic plots show these network injuries in dark grey color at baseline 1010, week 2 (MADRS=24) 1030 and week 8 (MADRS=24) of therapy 1050. The presence of abnormal network fragmentation can be observed in the right primary motor region at baseline 1020, after two weeks 1040 and after eight weeks of antidepressant therapy 1060.

FIG. 11 displays topographic plots of normalized network fragmentation for a patient with Parkinson and severe motor disability. The network injury can be observed in the right primary motor cortex BA4 and BA5, 1110 and centro-parietal cortex 1120. Sagittal view shows that the network injury extends from the primary motor cortex to deep subcortical structures including the right thalamus 1130.

FIG. 12 displays sagittal transparent view that show the presence of injured brain circuitry in centro-parietal regions 1220 and subcortical regions that include thalamic nuclei 1210 for a selected patient with Parkinson and severe motor disability. Frontal transparent view shows the presence of injured brain circuitry in centro-parietal regions 1240 and subcortical regions that include thalamic nuclei 1230.

### DETAILED DESCRIPTION OF THE INVENTION

The next description provides details and examples to provide an understanding of the patent. The invention can be presented in different forms and not all unnecessarily details have been shown or described to avoid obscuring the invention. The drawings and specifications are explanatory, rather than restrictive.

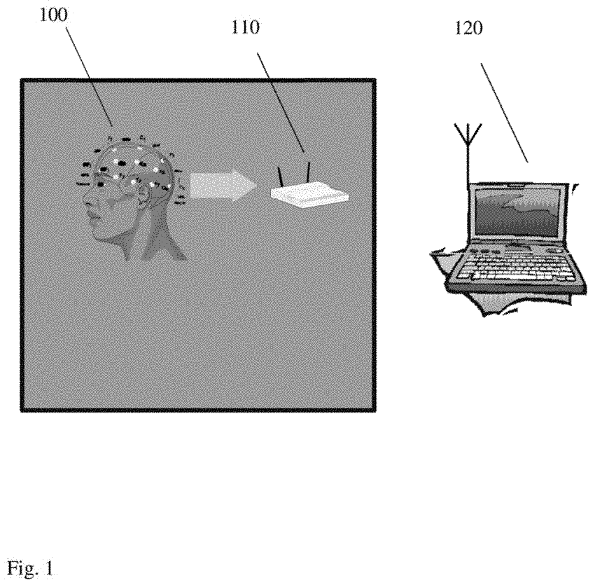

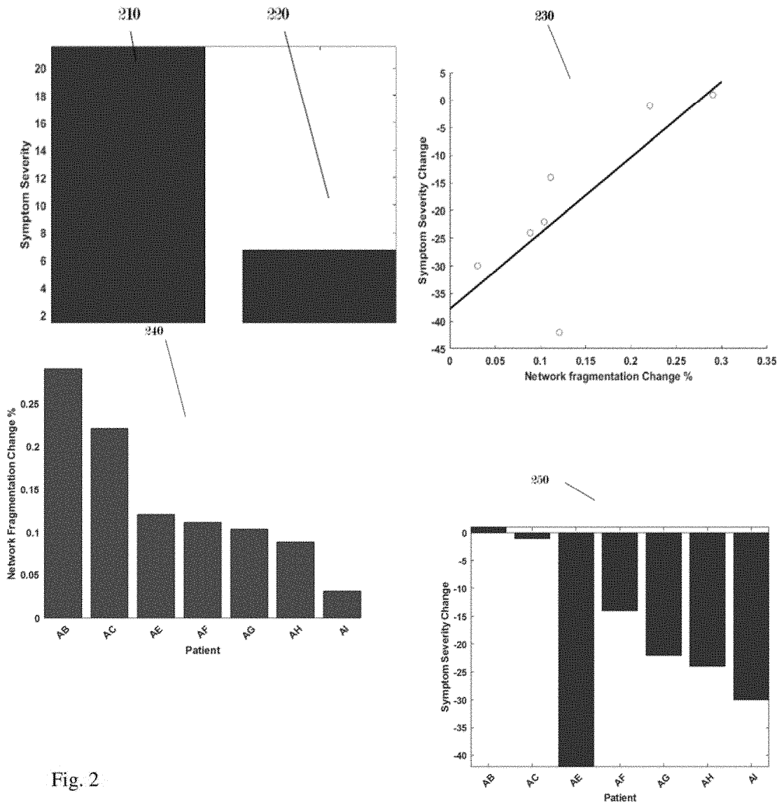

FIG. 1 shows a general illustration of the system of the present invention according to an example implementation. The system includes: an electroencephalograph (EEG) 110, a computing system 120 equipped with wireless transmission of information connected to a portable personal computer 130, or smartphones and wearable devices e.g. Google Glass The brain dysfunction can be any brain damage that leads to impairment of one or many specific functions that contribute to conscious experience, movement or posture dysfunction. In an embodiment of the invention network fragmentation. a connectivity measure can be estimated based on the inverse of dynamic cross-entropy (DCE) using resting state (eyes closed) EEG data recordings. By definition the term network injury refers to a brain dysfunction revealed based on estimated network fragmentation values. Compared to Innate resting-state Network Fragmentation (INF) determined in healthy subjects abnormal, exceedingly high or extremely low levels of network fragmentation define network injuries. FIG. 2 shows the drop of symptom severity between 1-week post TBI 210 and 1-year post TBI, 220. Symptom severity from the Sports Concussion Assessment Tool (SCAT3) was used for TBI assessment (Chin et al., 2016). The decrease of maximum value of network fragmentation is statistically significantly correlated with the reduction of symptom severity 230. Sorted values of percentage increase of maximum value of network fragmentation one year after TBI are shown for each subject 240. The reduction of symptom severity of each subject one year post-TBI is represented by bars 250.

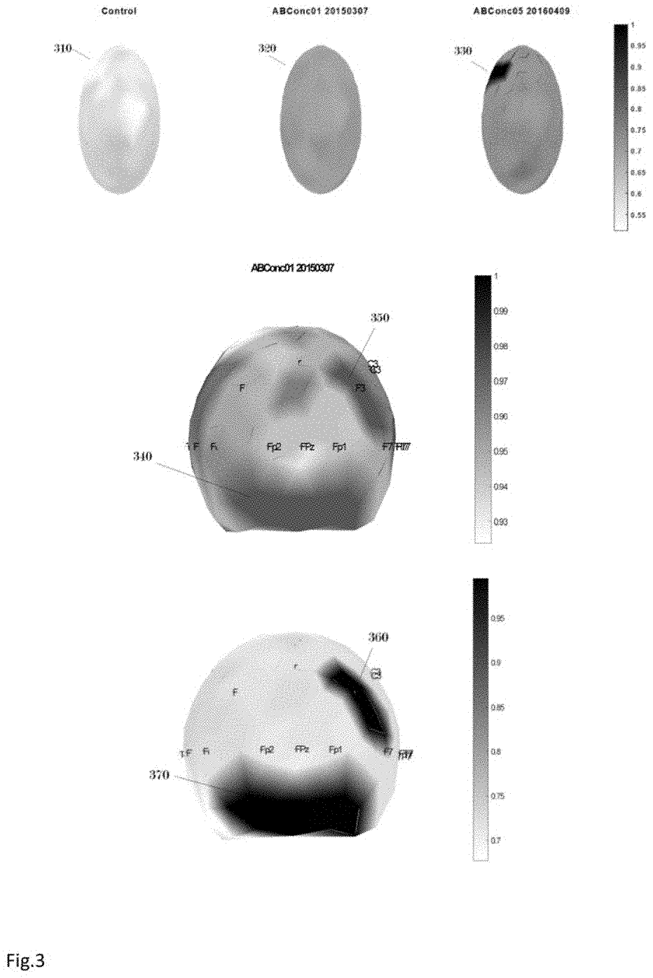

In another embodiment of the invention, NFMs are used to identify and detect the location of injured networks in traumatic brain injury. The NFMs were topographically plotted to show the difference between 1-week post TBI and 1-year post TBI, see FIG. 3. Mental health problems are generated by network dysfunctions (injuries) located in the brain regions with high, abnormal network fragmentation where the brain becomes randomly, chaotically connected with other brain regions. Such regions can be observed to occur and develop in some subjects after a mild traumatic brain injury.

FIG. 3 displays a frontal view of mean NFM of control subjects 310 and NFM of subject AB with traumatic brain injury at 1-week 320 and one year post-TBI 330. At 1-week after TBI, the presence of network injuries can be observed in the frontopolar 340 and supraorbital regions including left DLPFC 350. The increase of network fragmentation with 29% in the left DLPFC 360 and orbitofrontal regions 370 one year post-TBI inhibits functional recovery. Symptom severity increases from 3 to 4 one year post concussion. These mild injuries presented in FIG. 3 are from a hockey player. The helmet protects for possible skull fractures. however, it does not protect for brain injuries. The term “network injury” refers to a brain dysfunction revealed based on estimated network fragmentation which is abnormally increased or decreased compared to INF.

In this case detected network dysfunctions (injuries) behave no different than Phineas Gage injury (Ratiu et al., 2004). Psychiatric, mental or neurological dysfunctions are clinically expressed as pathological disconnection or over-connection patterns that can be easily detected in NFMs. The Innate resting-state Network Fragmentation (INF) is the representation of topographic map of mean network fragmentation of control subjects as shown in 330. This representation can be regarded as a reference of network interactions within a healthy brain. The INF is used as reference for further comparisons to determine if a given brain map presents network dysfunctions (injuries).

In another embodiment, the system and methods described herein may be used to provide precise therapy. Personalized therapy consist of non-invasive or invasive therapy, drug therapy or any other form of therapy that targets the location of brain dysfunctions which are detected using complexity measures (e.g. network fragmentation). The therapy becomes individualized, precise if it targets and removes network injuries (abnormal increased or decreased network fragmentation) and leads to a significant drop in symptom severity. The topographic representation of NFM shows regions with high, abnormal values where network injuries are located in case of different brain conditions. The stimulation or drug therapy has to target detected locations and their extensions to remove network dysfunctions (injuries).

Once the location of network dysfunction (injury) is known the physician can provide precise, personalized therapy. Different types of therapy such as infrared light, ultrasound, electromagnetic stimulation will generate a desired biological response in any of a variety of medical conditions including but not limited to mental health conditions (e.g. chronic depression, traumatic brain injury (TBI), chronic traumatic encephalopathy (CTE). autism spectrum disorders (ASD), post-traumatic stress disorder (PTSD), Alzheimer's, dementia, Parkinson. tinnitus, etc.) or various pain conditions if the therapy targets the regions with increased network fragmentation. Topographic representation of NFMs are used to monitor and evaluate the effect of therapy and can be correlated with different depression scales (see, Hawley et al., 2013).

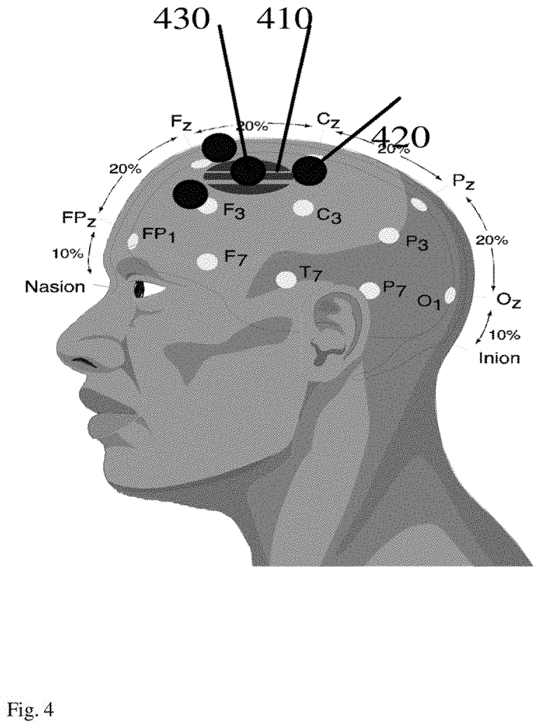

FIG. 4 shows an example where the proposed method uses the map of network fragmentation to locate the extension of network dysfunction (injury) 410 and determine the positioning of the built-on electrodes 420 to provide non-invasive stimulation. In order to maximize treatment benefit of non-invasive stimulation one electrode needs to be in the focal point of the network injury 440. The generated current by the non-invasive system 430 will flow within the brain between the electrode located in the middle 440 and other electrodes 420. The presented example does not restrict the position or the configuration of electrodes and different arrangement of electrodes can be considered in order to target the injured networks.

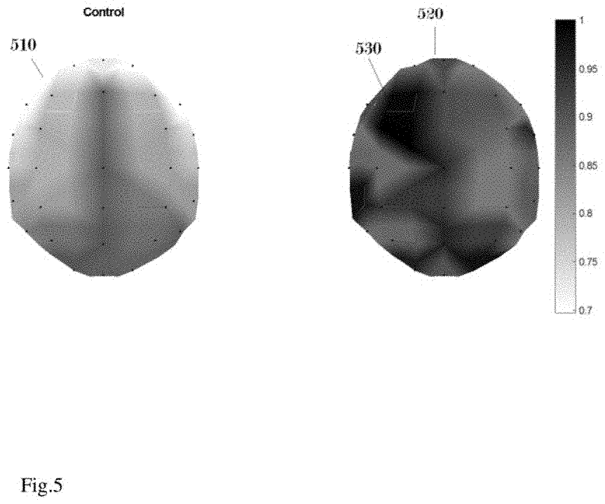

In another embodiment brain maps of network fragmentation can be used to compare control subjects and patients with mental health problems. FIG. 5 displays the mean of network fragmentation of 15 control subjects 510. The network fragmentation of selected remitter with mild depression at baseline (HDRS=17) is shown before therapy in 520. The patient has increased network fragmentation in the left dorsolateral prefrontal region (DLPFC). The network dysfunction (injury) is located in the left DLPFC 530, and displayed in dark gray color. The presence of network dysfunction (injury) can be rapidly identified by comparing the brain map 520 and INF brain image 510. One embodiment shows the effect of 10 Hz repetitive TMS (rTMS) therapy on NFM, e.g., that targets the foci of network injury, in the left dorsolateral prefrontal cortex (DLPFC).

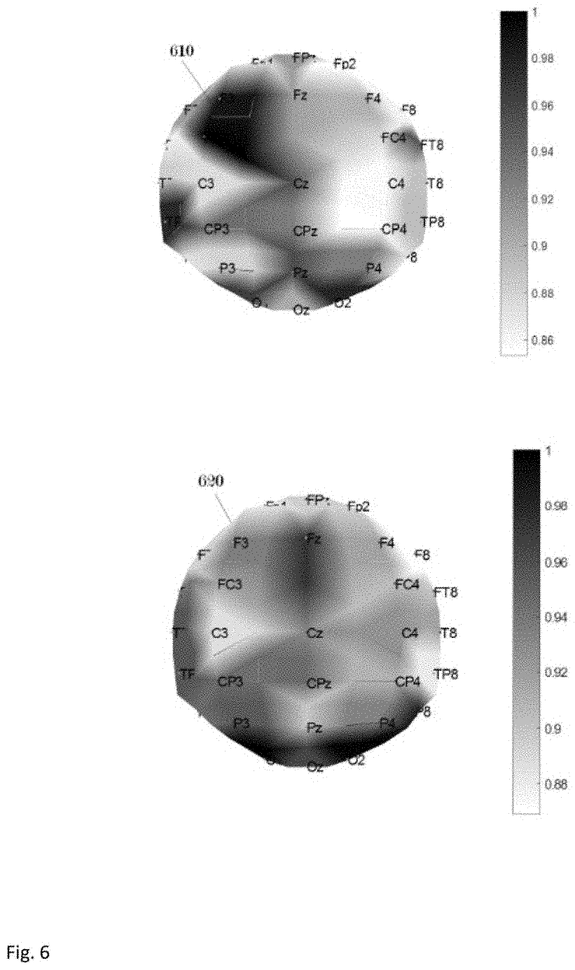

FIG. 6 displays the topographic representation of NFM in selected remitter with mild depression (HDRS=17) before therapy at baseline. The presence of network dysfunction (injury) can be observed in dark grey color in the left dorsolateral prefrontal cortex 610. Three months later after the rTMS therapy the network injury cannot be detected in the left DLPFC, see the light grey color at F3 site 620. The region with increased network fragmentation was removed from the left DLPFC and depression symptoms were lifted (HDRS=2) after non-invasive stimulation. Therefore, to achieve remission the therapy has to target the regions with abnormal network fragmentation and remove their presence.

In another embodiment the presented technique and NFM are used to monitor the effects of drug therapy.

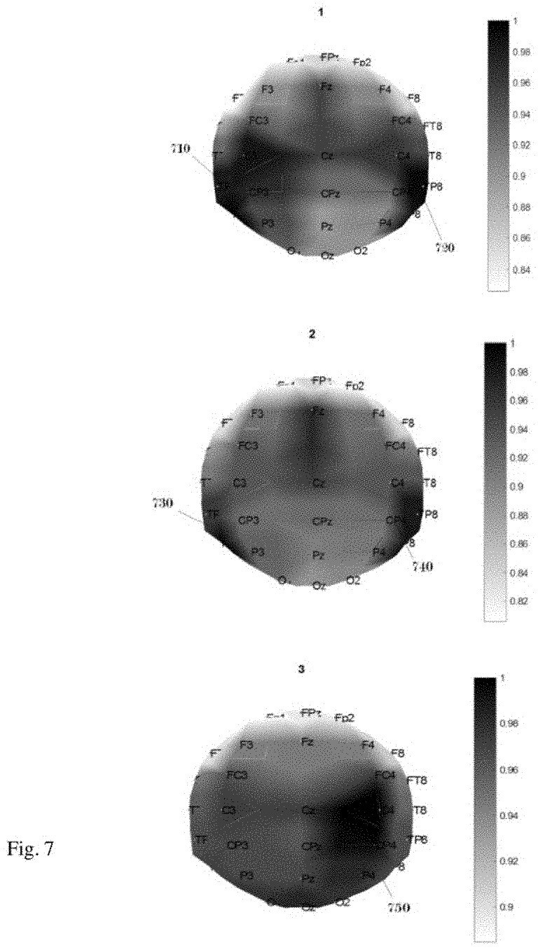

FIG. 7 displays a baseline NFM of a selected non-remitter. At baseline, before therapy network dysfunctions (injuries) can be observed in dark grey color in the left parieto-temporal region 710 and right parieto-temporal region 720. The severity of depression decreases from MADRS=35 at baseline to MADRS=27 after two weeks of drug therapy. Also, a drop of network fragmentation and a slight shift of network injury to parietal regions 730 and 740 can be observed. After 8 weeks of escitalopram therapy the severity of depression drops further (MADRS=14) and network injury has shifted to the right primary motor cortex 750. The severity of depression decreased from MADRS=35 at baseline to MADRS=27 at week 2 and MADRS=14 after 8 weeks of escitalopram, however, MADRS increased later at week 14 (MADRS=21). This later increase of symptom severity is related to the new location of network dysfunction (injury) in the right primary motor cortex at electrode C4. Importantly, this example shows that drug therapy can decrease network fragmentation and also change the location of network dysfunctions (injury) in the brain. Such spatial shift of the network dysfunction (injury) has consequences on symptom severity changes. or the occurrence of side effects after therapy. Therefore, based on NFM the occurrence of side effects can be predicted and avoided in time.

In another embodiment NFMs are used to predict and explain the effects of drug therapy. Linear mixed-effects models (see, Pinherio and Bates, 1996; Bates et al., 2014) are used to determine the brain regions for which drug therapy has a significant effect in major depressive disorder. The relationship between the scalar variable represented by the drop of Montgomery-Åsberg Depression Rating Scale (MADRS), and explanatory variables represented by changes of network fragmentation were estimated at each EEG electrode after two months of drug administration. The significance of the F-test indicates if linear regression model provides a better fit to the data than the model that contains no explanatory variables. The linear mixed-effects model was corrected for confounding factors such as age, sex.

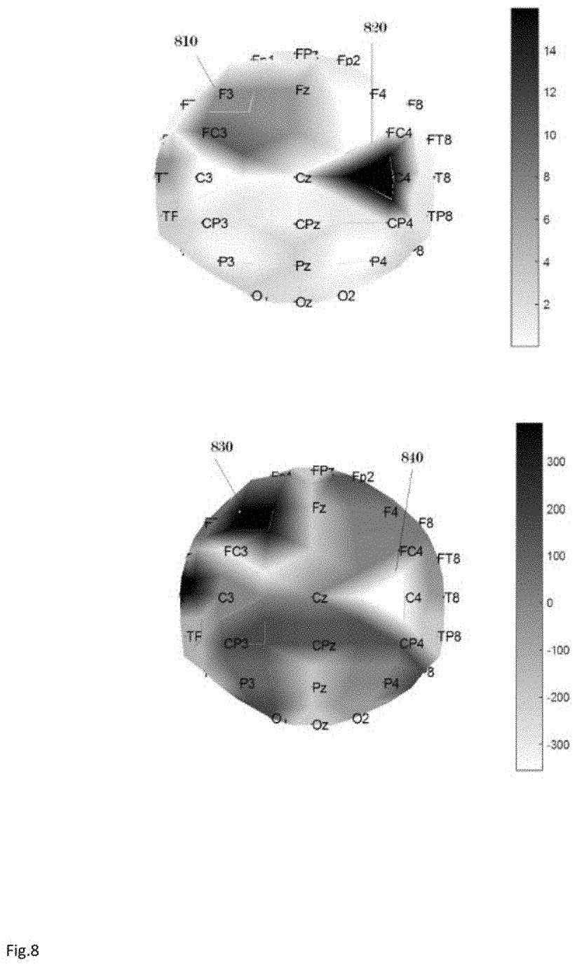

FIG. 8 displays a topographic plot of F-test statistic of linear regression that shows specific brain regions in dark grey color in the left DLPFC 810, right primary motor region 820 where there is a statistically significant relationship between the changes of network fragmentation and the drop of symptom severity eight weeks after escitalopram administration. A decrease of depression symptoms occurs in the next two months after selective serotonin reuptake inhibitors (SSRI) administration (e.g. escitalopram) if network dysfunctions are located in brain regions with high positive regression coefficients represented in dark grey color in the left DLPFC 830 and an increase of depression symptoms can be observed in patients with network dysfunctions (injuries) located in brain regions with high negative values of regression coefficients represented in light grey color in the right primary motor cortex 840. This analysis explains why the severity of depression has increased in the selected patient for which SSRI therapy has shifted the location of network injury to the right primary motor cortex at electrode C4 as presented in FIG. 7. Since the 1990s we know that SSRIs can in time induce movement disorders including dystonia, dyskinesia, tardive dyskinesia and Parkinsonism (Fitzgerald and Healy, 1995; Leo, 1996; Gerber and Lynd, 1998). To our knowledge all previous brain mapping analyses were blind in showing this issue, however they were revealed by many studies regarding SSRI effects (Fitzgerald and Healy, 1995). Therefore, NFM can be used to determine the location of abnormal network fragmentation and predict which patients have high risk to develop motor dysfunctions if SSRI therapy is continued.

A similar linear regression technique can be used to determine the effects of different drugs in Parkinson, epilepsy or Alzheimer's disease. The Unified Parkinson's Disease Rating Scale (UPDRS) in Parkinson (Goetz et al., 2008). seizure rating scales in epilepsy or mini-mental state examination (MMSE) in Alzheimer. see (Kurlowicz & Wallace 1999, Robert et al., 2010) or similar measures will be used instead of MADRS.

The proposed method and system to locate the network injury can be extended to patients with epilepsy, Parkinson and Alzheimer's. The brain mapping allows to detect the regions and brain circuitry that need to be therapeutically targeted either using drug therapy or have to be invasively or no-invasively stimulated. Mental state examination (MMSE) can be used in case of Alzheimer (Kurlowicz Wallace 1999) or similar measures. The presented method uses statistical measures, in combination with linear regression, NFM and severity scales to provide the regions that have to be therapeutically targeted either by using drugs or brain stimulation in case of very different brain dysfunctions.

The injury with a large iron rod of Phineas Gage has been at the origin of cerebral functions localization widely presented by physicians and anatomists (Ratiu et al., 2014) and detected network injuries behave no different than Phineas Gage. Importantly, based on the location of such network injuries different psychiatric and neurological disorders are triggered. Therefore, the system and methods described herein can be used to provide diagnostic in case of different brain conditions or used as an adjunct to standard clinical practice.

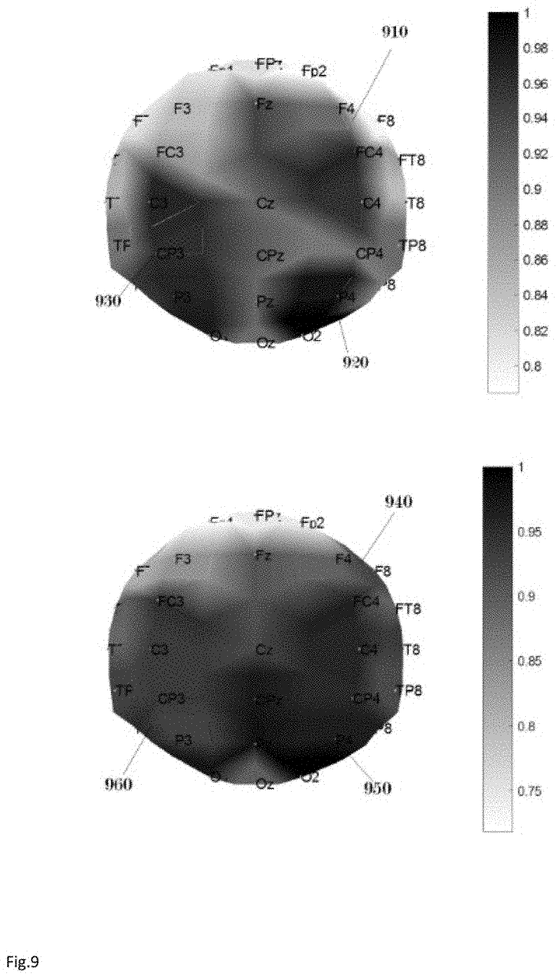

Another example shows that non-invasive stimulation of healthy brain regions can increase the severity of depression. Having identified the “injured” networks before therapy is highly important since the stimulation of healthy regions can increase symptom severity of depression. FIG. 9 displays a topographic representation of network fragmentation of a selected non-remitter with severe depression (HDRS=25) at baseline. The injured networks can be observed in the right DLPFC 910, right occipito-parietal region 920, see the dark grey region at electrodes: 4, Fc4, P4 and O2 and left primary motor region 930. The stimulation of left DLPFC which follows FDA approved protocol for TMS targets a healthy region in this case. Three months after TMS stimulation injured networks can be detected in the same brain regions in the right DLPFC 940, right occipito-parietal region 950, see the dark grey region at electrodes: F4, c4, P4 and O2 and left primary motor region 960. The stimulation of the healthy region (left DLPFC at F3 site) has worsened the severity of depression (HDRS=31). This example shows that the proposed system and methods can be used to improve the efficacy of TMS and electroconvulsive therapy (ECT) by displaying the topographic locations of injured networks in each patient before ECT is performed. The method and system can be used to determine which electrode placement (bitemporal, right unilateral or bifrontal) offers better efficacy and less cognitive impairment after therapy in each patient. Importantly, in addition to detecting network dysfunctions the new method can be used to select the appropriate drug dosage to provide therapy for MDD patients.

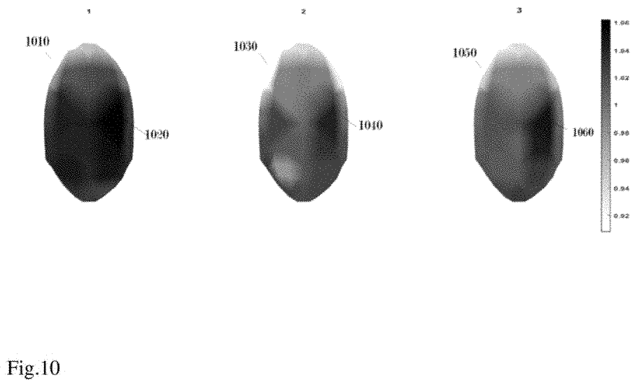

If the network injury is located in the right primary motor region at electrode C4 the severity of depression will increase if antidepressants are administered. The effects of drug therapy can be monitored after drug administration by mapping network fragmentation (see, FIG. 10). An example of a patient with high, abnormal network fragmentation located in the right primary motor cortex, at electrode C4 is presented in FIG. 10. The severity of depression increases from MADRS=22 at baseline to MADRS=24 after escitalopram administration. The topographic plots show network injuries in dark grey color at baseline 1010, week 2(MADRS=24) 1030 and week 8(MADRS=24) of therapy 1050. The presence of abnormal network fragmentation can be observed in the right primary motor region at baseline 1020, after two weeks 1040 and after eight weeks of antidepressant therapy 1060. The severity of depression increases after two weeks of antidepressant therapy from MADRS=22 to MADRS=24 and remains the same after eight weeks of therapy. This outcome after SSRI therapy reinforces the results of linear regression presented above. Therefore, NFM can be used to avoid therapeutic blunders either in non-invasive stimulation or drug therapy.

In addition, most techniques used for neurofeedback therapy are not reliable. Too often such techniques pick changes of noise levels and the brain maps show increased or decreased brain activity. The determination of brain regions that have to be targeted by therapy is unreliable due to volume conduction and noise presence in EEG recordings.

The assumptions about the nature of signals or electrical sources (e.g., number of sources. stationarity, smoothness, correlation, sparsity. spatial extent constraints, etc.) and the presence of spurious connectivity due to volume conduction make most techniques weak in detecting pathological network disconnection. They led to inconsistent, contradictory results, however, only lately these issues have been thoroughly recognized (see Anzolin et al., 2019).

In addition, the training effects of neurofeedback from one location spread to other electrode locations (brain regions). The proposed solution in this patent avoids using such techniques that have been found to have limited therapeutic potential (see, Gruzelier, 2014). Since the estimation of network fragmentation is immune to volume conduction and intrinsic noise the proposed solution can be used to improve the therapeutic potential of neurofeedback systems by using a modulating sound. The neurofeedback is applied after the computing system has determined for each individual subject the location and the extent of the brain regions that have to be stimulated based on resting-state activity recordings. Based on NFM the frequency of the sound is tuned to inform the patient when and whether the changes of network fragmentation occur in the desired direction in a brain region that was previously determined. If the patient is unable to learn how to change specific characteristics of connectivity non-invasive stimulation will be used to target the region with low-dose currents trans-cranially applied on the EEG electrode in specific, predetermined brain regions as presented above.

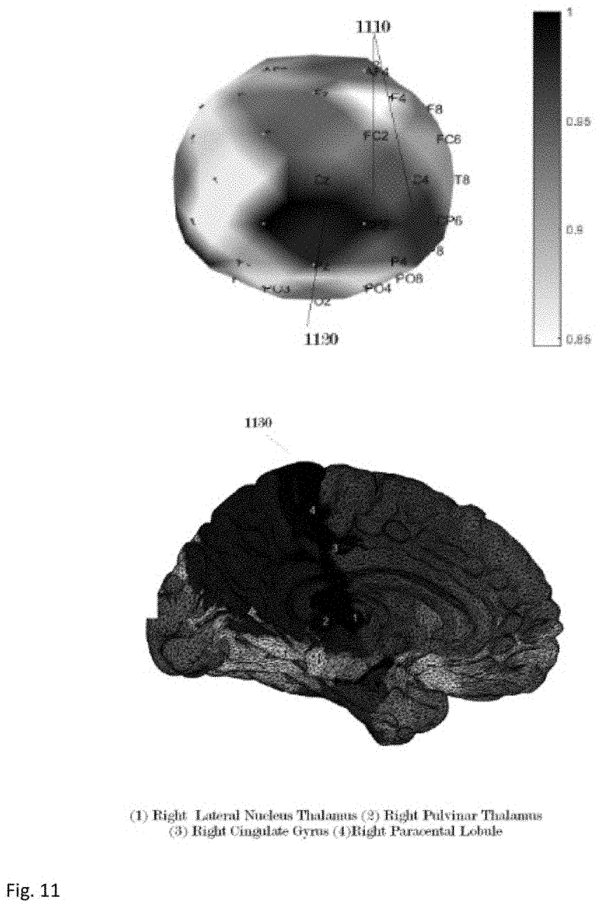

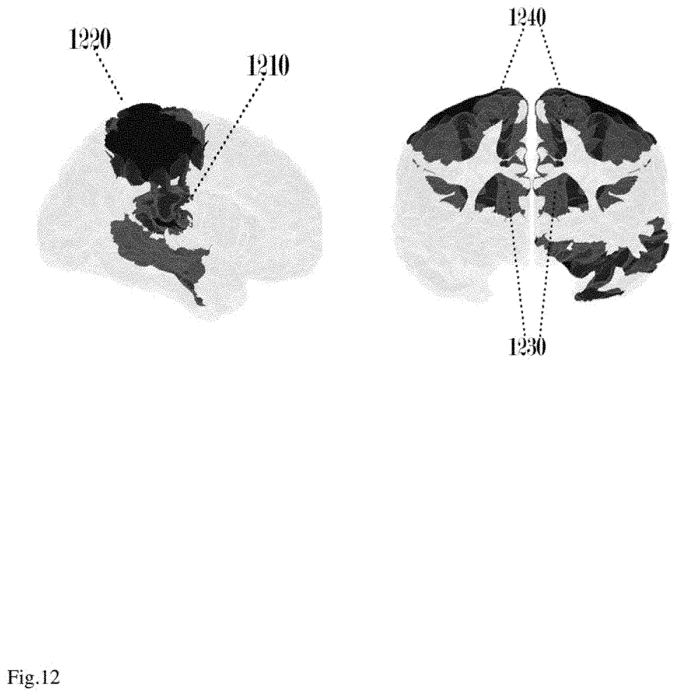

The present invention can be extended to locate the network injury within subcortical structures in patients with epilepsy, Parkinson and Alzheimer's disease based on NFM. The proposed method maps network fragmentation directly to the stereotaxic brain space. The acquired scalp positions of different EEG system the 10/20, 10/10 and 10/5 systems were registered to the Montreal Neurological Institute (MNI) stereotactic coordinates. Volume-based finite difference model (FDM) and triangulation are used to map network fragmentation and register EEG data to the stereotaxic brain space (Neuner et al., 2014; Aur and Jog, 2010). In addition, NFMs are used detect the location of injured regions within subcortical structure in case of different brain conditions that may include epilepsy, Alzheimer's disease or Parkinson as presented in FIG. 11 and FIG. 12.

Since the brain maps are registered to the Montreal Neurological Institute (MNI) stereotactic coordinates, they show which brain circuits are injured in each patient. The NFMs precisely locate the foci and the extension of network dysfunctions (injuries) within subcortical structures based on the estimated values of network fragmentation. In addition, 3D brain mappings and sagittal brain views can be used to understand the effects of therapy on network injury. Importantly, these brain maps indicate if appropriate levels of drug are in the brain, and if the administered drug targets the required injured region. The changes of drug concentrations in specific brain regions can be estimated based on changes that occur in the mapped networks by comparing longitudinal data as presented in FIG. 10.

In the examples provided herein, dynamic cross entropy is used without limiting the invention since other complexity measures could also be used to estimate dynamic fragmentation of the network e.g. Lyapunov exponent, algorithmic complexity measures such as Lempel-Ziv complexity, auto-mutual information, sample entropy. Tsallis entropy, approximate entropy, multiscale entropy (Vitanyi & Li, 1997; Mizuno et al. 2010) or fractal measures (Edgar, 1998; Zhao et al., 2016).

The theoretical framework, the model of computation that estimates network fragmentation is general. This model can be applied to detect network dysfunctions for different brain conditions and insights regarding network and injured neural circuits (Aur and Jog, 2010; Aur et al., 2011, Aur and Tuszynski, 2017). The regions with increased network fragmentation can be separated and shown in transparent views which allows to determine the extension of dysfunctional regions.

Targeting detected injured networks with non-invasive stimulation and electric or magnetic patterns will change the brain rhythms immediately. If non-invasive stimulation is periodically repeated it will plastically alter the molecular structure within neurons, synapses and glial cells (Aur and Jog, 2010, Aur et al., 2011, Aur et al., 2016) and remove network dysfunctions (injuries) and decrease the symptoms in case of different brain conditions. The new resulting brain structure after stimulation will generate new rhythms and restore the brain function. The application of non-invasive stimulation will eliminate abnormal levels of network fragmentation in patients with major depression as long as the correct brain region is identified and targeted by therapy.

For various brain conditions the system and the presented method provide brain maps which include the location of brain dysfunctions that have to be targeted by therapy to improve the brain function.

### EXAMPLES

### Example 1: Detection of Injured Networks in Parkinson with Network Fragmentation Maps

Network fragmentation quantifies the randomness of brain interactions estimated based on dynamic cross-entropy (DCE) values. Network injuries are located in brain regions with high, abnormal values of network fragmentation. Resting state EEG was recorded using a 32-electrode cap with Ag/AgCl electrodes and then amplified. The bandwidth of the amplifiers was between (0.016-500 Hz) and data was sampled at 1000 Hz. An additional 250 Hz low-pass band filter was used and the impedance at all recording electrodes was less than 5 kΩ. Horizontal eye movements and vertical eye movements were recorded with electrodes placed near the outer cantus of each eye respectively above and below the center of the left bottom eyelid. The common average reference is used to decrease the confounding effect of brain activity.

For each data set the raw EFG resting state data is split into segments of 1-second duration and segments that contain artifacts are detected and excluded from analysis. Since the presence of broadband high-frequency can be an indicator of electromyographic contamination to minimize the influence of artifacts an automatic deartifacting method that filters out regions with broadband high-frequency and widespread low-frequency power increase was used. Periods having activity three standard deviations away from the mean were removed from the data. On average 160 remaining segments of resting state data were used. A notch filter at 60 Hz was used to remove the line noise.

Network fragmentation is estimated as the inverse of dynamic cross entropy (DCE) in delta band and directly mapped to the stereotaxic brain space. The brain maps in FIG. 11 are from a selected patient with Parkinson that has severe motor disability. The network is injured in the right and in the left primary motor cortex Brodmann area BA4 and BA5, 1110 and centro-parietal cortex 1120. The sagittal view shows that the network dysfunction (injury) extends from the primary motor cortex to subcortical structures including the right thalamus 1130. The brain map indicates the region with high abnormal network fragmentation that has to be targeted by therapy either by administering drugs or deep brain stimulation to remove the network injury. Mapping network fragmentation to the Montreal Neurological Institute (MNI) stereotactic coordinates enables data integration across different subjects (Vatta et al., 2010).

### Example 2: Detection of Injured Networks in Parkinson with Network Fragmentation Maps

In another embodiment of the invention, the regions with increased network fragmentation can be separated. This novel method is used to identify the injured brain circuitry and presented in transparent views.

For the selected patient with Parkinson and severe motor disability FIG. 12 displays sagittal transparent view that shows the presence of injured brain circuitry in centro-parietal regions 1220 and subcortical regions that include thalamic nuclei 1210. Frontal transparent view shows the presence of injured brain circuitry in centro-parietal regions 1240 and subcortical regions that include thalamic nuclei 1230.

Once the location of injured brain network is known, either non-invasive stimulation or various drugs can provide therapy. The method can also be used to monitor the effects of therapy and measure the effect of therapy on injured networks, or if the drug has targeted the required region. Drug concentrations in specific brain regions can be estimated based on changes that occur in these networks by comparing the baseline reference of network fragmentation and gray (colored) scales over repeated cross-longitudinaldata.

### REFERENCES

Acharya, U. R., Hagiwara. Y., Deshpande, S. N., Suren, S., Koh, J. E. W., Oh. S. L., . . . & Lim, C. M. (2018). Characterization of focal EEG signals: a review. Future Generation Computer Systems.
Anzolin, A., Presti, P., Van De Steen, F., Astolfi, L., Haufe, S., & Marinazzo, D. (2019). Quantifying the effect of demixing approaches on directed connetivity estimated between reconstructed EEG sources. Brain topography, 1-20.
Aur, D.,& Jog, M. S. (2010). Neuroelectrodynamics: understanding the brain language IOS Press.
Aur, D., Jog, M., & Poznanski, R. R. (2011). Computing by physical interaction in neurons. Journal of integrative Neuroscience, 10(04), 413-422.
Aur, D., & Vila-Rodriguez, F. (2017). Dynamic Cross-Entropy. Journal of neuroscience methods, 275, 10-18.
Aur, D., & Jog. M. S. (2018). Focal and Diffuse Injuries on Dynamic Network Patterns are at the Origins of Major Depression. submitted, August 2018
Aur, D., Munjal V., Muller A., Viji-Babul N., (2018). Evidence of Brain Network Fragmentation 1-year Post Concussion in Adolescent Athletes: A Pilot Study, Poster session presented at the GF Strong Rehab Research Day.
Aur D. 2011, Understanding the Physical Mechanism of Transition to Epileptic Seizures, Journal of Neuroscience Methods. Volume 200, Issue 1, 30 August pp 80-85.
Aur, D., Pang, C., Brenner C, Blumberger, D., Downar, J., Lam R, & Vila-Rodriguez, F, 2017, TMS Reshapes Spatial Distributions of Resting State Beta in MDD patients, OHBM.
Aur. D., Toyoda, I., Bower, M. R., & Buckmaster, P. (2013). Seizure prediction and neurological disorder treatment. U.S. Pat. No. 8,600,513. Washington, D.C.: U.S. Patent and Trademark Office.
Biscay, R. J., Bosch-Bayard, J. F., Pascual-Marqui, R. D. 2018. Unmixing EEG Inverse solutions based on brain segmentation Frontiers in Neuroscience 12(May), 325
Chin, E. Y., Nelson, L. D., Barr, W. B., McCrory, P. & McCrea, M. A. (2016). Reliability and validity of the Sport Concussion Assessment Tool-3(SCAT3) in high school and collegiate athletes. The American journal of sports medicine, 44(9), 2276-2285.
Edgar, G. A. (1998). Fractal Measures. In Integral, Probability, and Fractal Measures (pp. 1-67). Springer, New York, N.Y.
Fischell, Robert E.; Fischell, David R. (Neuro Pace, Inc.) 2001 Integrated system for EEG monitoring and electrical stimulation with a multiplicity of electrodes, Patent umber U.S. Pat. No. 6,230,049
Fitzgerald, K., & Healy, D. (1995). Dystonias and dyskinesias of the jaw associated with the use of SSRIs. Human Psychopharmacology: Clinical and Experimental, 10(3), 215-219
Gerber, P. E., & Lynd, L. D. (1998). Selective serotonin-reuptake inhibitor-induced movement disorders. Annals of Pharmacotherapy, 32(6), 692-698.
Grech, R., Cassar, T., Muscat, J., Camilleri, K. P., Fabri, S. G., Zervakis, M., . . . & Vanrumste, B. (2008). Review on solving the inverse problem in EEG source analysis. Journal of neuroengineering and rehabilitation, 5(1), 25.
Goetz, C. G., Tilley, B. C., Shaftman, S. R., Stebbins, G. T., Fahn, S., Martinez-Martin, P., . . . & Dubois, B. (2008). Movement Disorder Society-sponsored revision of the Unified Parkinson's Disease Rating Scale (MDS-UPDRS): Scale presentation and clinimetric testing results. Movement disorders, 23(15), 2129-2170.
Gruzelier, J. H. (2014). EEG-neurofeedback for optimising performance. I: a review of cognitive and affective outcome in healthy participants. Neuroscience & Biobehavioral Reviews, 44, 124-141.
Hawley, C. J., Gale, T. M., Smith, P. S. J., Jain, S., Farag, A., Kondan, R., . . . & Graham, J. (2013). Equations for converting scores between depression scales (MÅDRS, SRS. PHQ-9 and BDI-II): good statistical, but weak idiographic, validity. Human Psychopharmacology: Clinical and Experimental, 28(6), 544-551.
Klotz, J. M., 1993, Topographic EEG mapping methods. Cephalalgia 13(1), pp. 45-52 Kurlowicz, L., & Wallace, M. (1999). The mini-mental state examination (MMSE). Journal of gerontological nursing, 25(5), 8-9.
Lenoir, C., Jankovski, A., & Mouraux, A. (2018). Anodal transcutaneous spinal direct current stimulation (tsDCS) selectively inhibits the synaptic efficacy of nociceptive transmission at spinal cord level. Neuroscience, 393, 150-163.
Michel, C. M., Murray, M. M., Lantz, G., Gonzalez, S., Spinelli, L., & de Peralta, R. G. (2004). EEG source imaging. Clinical neurophysiology, 115(10), 2195-2222.
Mitchell. P. B. (2000). Therapeutic drug monitoring of psychotropic medications. British journal of clinical pharmacology, 49(4), 303-312.
Mizuno, T., Takahashi, T., Cho, R. Y., Kikuchi, M., Murata. T., Takahashi, K., & Wada, Y. (2010). Assessment of EEG dynamical complexity in Alzheimer's disease using multiscale entropy. Clinical Neurophysiology, 121(9), 1438-1446.
Neuner, I., Arrubla, J., Werner, C. J., Hitz, K., Boers, F., Kawohl, W., & Shah, N. J. (2014). The default mode network and EEG regional spectral power: a simultaneous fMRI-EEG study. PLoS One, 9(2), e88214.
Oostenveld, R., Fries, P., Maris, E., & Schoffelen, J. M. (2011). FieldTrip: open source software for advanced analysis of MEG, EEG, and invasive electrophysiological data. Computational intelligence and neuroscience, 2011, 1.
Paris, J. (2014). The mistreatment of major depressive disorder. The Canadian Journal of Psychiatry, 59(3), 148-151.
Ratiu, P., Talos, I. F., Haker, S., Lieberman. D., & Everett, P. (2004). The tale of Phineas Gage, digitally remastered. Journal of neurotrauma, 21(5), 637-643.
Robert, P., Ferris, S., Gauthier, S., Ihl, R., Winblad, B., & Tennigkeit, F. (2010). Review of Alzheimer's disease scales: is there a need for a new multi-domain scale for therapy evaluation in medical practice?. Alzheimer's research & therapy, 2(4), 24.
Van Veen, B. D., Van Drongelen, W., Yuchtman, M., & Suzuki, A. (1997). Localization of brain electrical activity via linearly constrained minimum variance spatial filtering. IEEE Transactions on biomedical engineering, 44(9), 867-880.
Vatta, F., Meneghini, F., Esposito, F., Mininel, S., & Salle, F. D. (2010). Realistic and spherical head modeling for EEG forward problem solution: a comparative cortex-based analysis. Computational intelligence and neuroscience, 2010, 13.
Vitanyi, P. M., & Li, M. (1997). An introduction to Kologorov complexity and its applications (Vol. 34, No. 10). Heidelberg: Springer.
Wang, J., Huang, H., Yao, Q., Lu, Y., Zheng, Q., Cheng, Y., . . . & Li, X. (2015). Simple and accurate quantitative analysis of 16 antipsychotics and antidepressants in human plasma by ultrafast high-performance liquid chromatography/tandem mass spectrometry. Therapeutic drug monitoring, 37(5), 649-660.
Zhao, G., Denisova, K., Sehatpour, P., Long, J., Gui, W., Qiao, J., . . . & Wang, Z. (2016). Fractal dimension analysis of subconical gray matter structures in schizophrenia. PloS one, 11(5), e0155415.

1. A brain mapping system that allows to provide personalized, precise therapy for a broad range of brain dysfunctions that includes:
a. An electroencephalograph (EEG) and a computing system (device) that maps the location and the extent of the brain regions that have to be therapeutically targeted;
b. The computing system determines the location of the brain region that have to be therapeutically targeted by mapping a complexity measure, e.g. network fragmentation to provide successful therapy for each patient;
c. The network fragmentation of control subjects is used as reference for further comparisons to determine if the network fragmentation map of a given subject displays network dysfunctions;

2. The system as set forth in claim 1, wherein said it includes a computing device with display and allows physicians and technicians to identify before therapy which brain regions have to be therapeutically targeted by drugs, invasive or non-invasive stimulation;
a. The presence of network dysfunction (injury) can be rapidly identified by comparing the innate resting-state network fragmentation with subject's network fragmentation map;
b. Based on network fragmentation maps the occurrence of side effects can be predicted and avoided in time;
c. Based on network fragmentation maps the patients that have high risk to develop motor dysfunctions if serotonin reuptake inhibitor therapy is continued and can be identified;
d. The brain mapping system reveals specific locations of network dysfunctions in case of very different brain dysfunctions (epilepsy, major depression, stroke, traumatic brain injury, Alzheimer's, Parkinson, multiple sclerosis, Tourette syndrome; tinnitus; fibromyalgia);
e. The brain mapping system can identify a broad range of brain dysfunctions at an early stage;
f. The therapy that targets and removes brain injuries (network dysfunctions) is individualized, precise and leads to a drop in symptom severity;

3. The system as set forth in claim 1, wherein said it can be used to monitor the progress of therapy, determine deep brain structures that are injured and evaluate the clinical value of drug products;
a. The brain maps predict desirable and undesirable effects of treatments e.g. serotonin reuptake inhibitor therapy;
b. Network fragmentation maps precisely locate the foci and the extension of network injuries within subcortical structures based on estimated values of network fragmentation;
c. Network fragmentation maps are used to avoid therapeutic blunders either in non-invasive stimulation or drug therapy.
d. Network fragmentation or measures of complexity are directly mapped to stereotaxic brain space registered to the Montreal Neurological Institute (MN) by using volume-based finite difference model (FDM) and triangulation;

4. The system as set forth in claim 1, wherein said network fragmentation maps are used to determine the location of abnormal network fragmentation and predict which patients have high risk to develop motor dysfunctions if serotonin reuptake inhibitor therapy is continued.
a. Network fragmentation maps are used to adjust and tailor the therapy that may include noninvasive, invasive stimulation and/or drug therapy until specific characteristics of connectivity determined based on complexity measures are restored and brain dysfunctions or network injuries cannot be detected;
b. The neurofeedback stimulation requires active patient participation in the therapy by reinforcing the change in the brain regions with abnormal network fragmentation.
